(19) United States
(12) Patent Application Publication
(10) Pub. No.: US 2024/0315347 A1
(43) Pub. Date: Sep. 26, 2024

(54) ATOMIZATION DEVICE AND ELECTRONIC CIGARETTE

(71) Applicant: Luxshare Precision Industry Company Limited, Shenzhen (CN)

(72) Inventors: Huabing LI, Shenzhen (CN); Zhongyuan LAI, Shenzhen (CN); Chunde XIAO, Shenzhen (CN); Hongbing YIN, Shenzhen (CN); Zijun WANG, Shenzhen (CN); Xinnan ZHOU, Shenzhen (CN); Honglei BAN, Shenzhen (CN)

(21) Appl. No.: 18/377,981

(22) Filed: Oct. 9, 2023

(30) Foreign Application Priority Data
Mar. 23, 2023 (CN) 2023103563479

(51) Int. Cl. A24F 40/51; A24F 40/10; A24F 40/44; A24F 40/46; A61B 5/0205; A61B 5/145; H01M 50/202; H01M 50/247; H02J 7/00; H05B 3/22

(52) U.S. Cl. CPC A24F 40/51; A24F 40/10; A24F 40/44; A24F 40/46; A61B 5/0205; A61B 5/14542; H01M 50/202; H01M 50/247; H02J 7/0045; H05B 3/22; A61B 5/02438; H01M 2220/30

(57) ABSTRACT

Provided is an atomization device and an electronic cigarette. The atomization device includes a housing, an atomization module, a functional module and a power storage battery module. An atomization liquid is stored in the housing. The atomization module is disposed within the housing, the atomization module includes an electrically conductive heating circuit, and the electrically conductive heating circuit is capable of heating and atomizing the atomization liquid when the electrically conductive heating circuit is energized. The functional module includes a gas component detection unit. The power storage battery module is electrically connected to the functional module and capable of supplying power to the functional module. The power storage battery module is electrically connected to the electrically conductive heating circuit. While the electrically conductive heating circuit is energized, the power storage battery module is charged.

### CROSS-REFERENCE TO RELATED APPLICATIONS

This application claims priority to Chinese Patent Application No. 2023103563479 filed Mar. 23, 2023, the disclosure of which is incorporated herein by reference in its entirety.

### TECHNICAL FIELD

This disclosure pertains to the technical field of electronic cigarettes, and more particularly, relates to an atomization device and an electronic cigarette.

### BACKGROUND

The atomization device is a device for atomizing test solution, and is an important component of an apparatus such as an air humidifier, a medical atomization device, or an electronic cigarette.

Currently, to realize multifunction of an atomization device, some atomization devices added with other functions (such as a gas component detection function) are emerged on the market. However, the atomization device generally supplies power to other functional modules while performing atomization in conjunction with the battery module, that is, the other functions can be realized only when the atomization is being performed, resulting in monotone and inflexible usage method and poor user experience.

Therefore, there is an urgent need for an atomization device and an electronic cigarette to address the above-mentioned technical issues.

### SUMMARY

The present disclosure provides an atomization device and an electronic cigarette to improve flexibility in use, thereby improving user experience.

To achieve this object, the following technical solutions are adopted in the present disclosure.

An atomization device includes a housing, an atomization module, a functional module and a power storage battery module.

An atomization liquid is stored in the housing.

The atomization module is disposed within the housing, and the atomization module includes an electrically conductive heating circuit, and the electrically conductive heating circuit is capable of heating and atomizing the atomization liquid while the electrically conductive heating circuit is energized.

The functional module includes a gas component detection unit.

The power storage battery module is electrically connected to the functional module and capable of supplying power to the functional module. The power storage battery module is electrically connected to the electrically conductive heating circuit, and while the electrically conductive heating circuit is energized, the power storage battery module is charged.

In an embodiment, the electrically conductive heating circuit includes a first electrode, an electrically conductive heat generation structure and a second electrode. One end of the first electrode is electrically connected to one end of the electrically conductive heat generation structure, and another end of the first electrode extends to an outer surface of the housing. One end of the second electrode is electrically connected to another end of the electrically conductive heat generation structure, and another end of the second electrode extends to the outer surface of the housing. A positive electrode of the power storage battery module is electrically connected to the first electrode, and a negative electrode of the power storage battery module is electrically connected to the second electrode.

In an embodiment, the power storage battery module includes a power storage battery, a first connection terminal and a second connection terminal. The power storage battery is electrically connected to the functional module. One end of the first connection terminal is electrically connected to the first electrode, and another end of the first connection terminal is electrically connected to a positive electrode of the power storage battery. One end of the second connection terminal is electrically connected to the second electrode, and another end of the second connection terminal is electrically connected to a negative electrode of the power storage battery.

In an embodiment, the first connection terminal includes a first sleeve portion and a first elastic piece. The first sleeve portion is sleeved on the first electrode. One end of the first elastic piece is connected to the first sleeve portion, and another end of the first elastic piece is welded to the positive electrode of the power storage battery.

The second connection terminal includes a second sleeve portion and a second elastic piece. The second sleeve portion is sleeved on the second electrode. One end of the second elastic piece is connected to the second sleeve portion, and another end of the second elastic piece is welded to the negative electrode of the power storage battery.

In an embodiment, an outer sidewall of the first sleeve portion is provided with multiple first through slots in communication with the inside of the first sleeve portion, one end of each of the first through slots extends to an end portion of one end of the first sleeve portion, and the first elastic piece is connected to another end of the first sleeve portion.

An outer sidewall of the second sleeve portion is provided with multiple second through slots in communication with the inside of the second sleeve portion, one end of each of the second through slots extends to an end portion of one end of the second sleeve portion, and the second elastic piece is connected to another end of the second sleeve portion.

In an embodiment, the first electrode includes a first connecting post and a first contact piece connected to one end of the first connecting post. One end, facing away from the first contact piece, of the first connecting post is electrically connected to the electrically conductive heat generation structure, the first contact piece is embedded on the outer surface of the housing, and the first connecting post is electrically connected to the positive electrode of the power storage battery module.

The second electrode includes a second connecting post and a second contact piece connected to one end of the second connecting post. One end, facing away from the second contact piece, of the second connecting post is electrically connected to the electrically conductive heat generation structure, the second contact piece is embedded on the outer surface of the housing, and the second connecting post is electrically connected to the negative electrode of the power storage battery module.

In an embodiment, the atomization module further includes a liquid infiltration member. The atomization liquid is disposed on one side of the liquid infiltration member, the electrically conductive heating circuit is connected to another side of the liquid infiltration member, and the liquid infiltration member is capable of being infiltrated by the atomization liquid.

In an embodiment, the housing includes a main body housing and a housing cover, and the housing cover is covered on the main body housing. One end of the liquid infiltration member is disposed in the housing cover, and another end of the liquid infiltration member is disposed in the main body housing. A storage chamber for storing the atomization liquid is disposed on a side, facing the main body housing, of the liquid infiltration member, and an atomization chamber is disposed on a side, facing the housing cover, of the liquid infiltration member. Each of the electrically conductive heating circuit, the functional module, and the power storage battery module is disposed in the atomization chamber.

In an embodiment the gas component detection unit, a blood oxygen detection unit and a heart rate detection unit are integrated in the functional module.

An electronic cigarette includes a power supply module, and further includes the above-described atomization device. The power supply module is detachably connected to the housing and is capable of supplying power to the electrically conductive heating circuit.

### BRIEF DESCRIPTION OF DRAWINGS

To illustrate the technical solution in embodiments of the present disclosure more clearly, the drawings used in description of the embodiments of the present disclosure are briefly described below. Apparently, the drawings described below merely illustrate part of the embodiments of the present disclosure, and those of ordinary skill in the art can also obtain other drawings on the basis of the contents in the embodiments of the present disclosure and theses drawings on the premise that no creative efforts are made.

FIG. 1 is a structure view of an atomization device according to an embodiment of the present disclosure;

FIG. 2 is a sectional view of the atomization device according to an embodiment of the present disclosure;

FIG. 3 is a first view showing partial structure of the atomization device according to an embodiment of the present disclosure;

FIG. 4 is a second view showing partial structure of the atomization device according to an embodiment of the present disclosure;

FIG. 5 is an exploded view showing partial structure of the atomization device according to an embodiment of the present disclosure;

FIG. 6 is an exploded view of the atomization device according to an embodiment of the present disclosure; and

FIG. 7 is a third view showing partial structure of the atomization device according to an embodiment of the present disclosure.

### REFERENCE LIST

1 housing
11 main body housing
12 housing cover
121 gas outlet
13 storage chamber
14 atomization chamber
15 gas channel
16 cylinder
2 atomization module
21 liquid infiltration member
22 electrically conductive heating circuit
221 first electrode
2211 first connecting post
2212 first contact pad
222 electrically conductive heat generation structure
223 second electrode
3 functional module
31 block
32 heart rate detection member
33 blood oxygen detection member
34 gas detection member
4 first sealing ring
5 second sealing ring
6 third sealing ring
7 fixation holder
71 limiting hole
72 limiting recess
8 residual liquid adsorbing layer
9 power storage battery module
91 power storage battery
92 first connection terminal
921 first sleeve portion
9211 first through slot
922 first elastic piece
93 second connection terminal

### DETAILED DESCRIPTION

Embodiments according to the present disclosure are described in detail hereinafter. Examples of the embodiments are illustrated in the drawings, where the same or similar reference numerals indicate the same or similar parts or parts having the same or similar functions. The embodiments described hereinafter with reference to the drawings are exemplary, and are intended to explain the present disclosure, rather than being construed as limiting the present disclosure.

In the description of the present disclosure, unless otherwise clearly specified and limited, the terms “jointed”, “connected” and “mounted” should be understood in a broad sense, for example, it can be a mounted connection, a detachable connection; it can be a mechanical connection or an electrical connection; it can be a direct connection or an indirect connection through an intermediary, and it can be the internal communication of two components or the interaction relationship between two components. Those of ordinary skill in the art can understand the specific meanings of the above terms in the present disclosure according to specific situations.

In the description of the present disclosure, unless otherwise expressly specified and limited, a first feature being “on” or “under” a second feature may include direct contact between the first and second features, and may also include the first and second features not in direct contact but in contact through another feature between them. Moreover, the first feature is “over”, “above” and “on” the second feature include that the first feature is directly above and obliquely above the second feature, or simply means that the first feature is horizontally higher than the second feature. The first feature is “beneath”, “below” and “under” the second feature include that the first feature is directly below and obliquely below the second feature, or simply means that the first feature has a lower level than the second feature.

Technical solutions of the present disclosure are further described hereinafter in conjunction with the drawings and the embodiments.

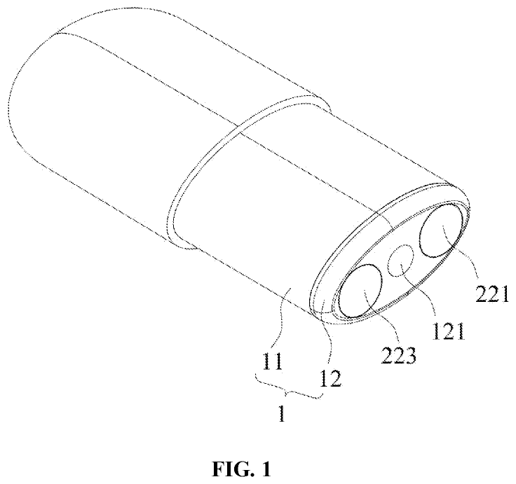

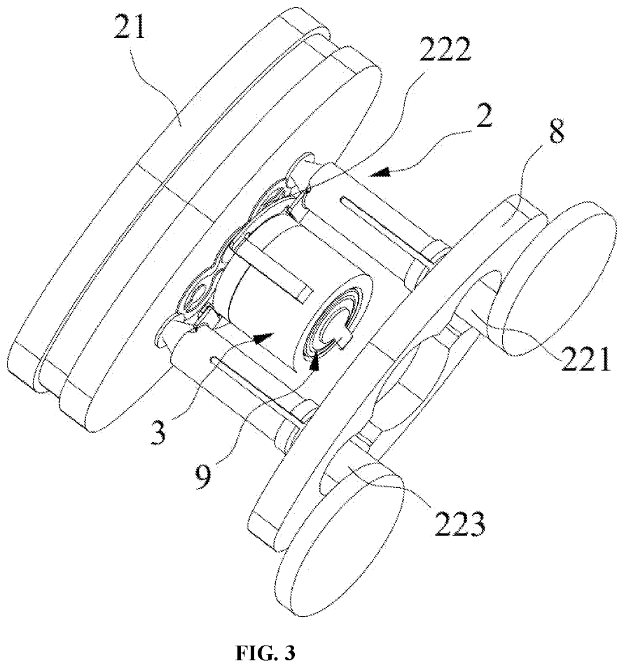

As shown in FIGS. 1 to 3, this embodiment provides an atomization device, which includes a housing 1, an atomization module 2, a functional module 3 and a power storage battery module 9. An atomization liquid is stored in the housing 1. The atomization module 2 is disposed within the housing 1, and includes an electrically conductive heating circuit 22, and the electrically conductive heating circuit 22 is capable of heating and atomizing the atomization liquid when energized. The functional module 3 includes a gas component detection unit. The power storage battery module 9 is electrically connected to the functional module 3, and can supply power to the functional module 3. The power storage battery module 9 is electrically connected to the electrically conductive heating circuit 22. When the electrically conductive heating circuit 22 is energized, the power storage battery module 9 is charged.

In the atomization device provided by this embodiment, when atomization is to be performed, the electrically conductive heating circuit 22 is energized to heat the atomization liquid to implement atomization, and meanwhile, the power storage battery module 9 is charged. After the electrically conductive heating circuit 22 is de-energized, the atomization ends, and the power storage battery module 9 with power stored can supply power to the functional module 3 separately. That is, the atomization device can detect the surrounding gas separately even when atomization is not being performed. Therefore, the functional module 3 can work independently, so that the atomization device can be used flexibly and the user experience is improved.

Specifically, the electrically conductive heating circuit 22 includes a first electrode 221, an electrically conductive heat generation structure 222 and a second electrode 223. One end of the first electrode 221 is electrically connected to one end of the electrically conductive heat generation structure 222, and another end of the first electrode 221 extends to an outer surface of the housing 1. One end of the second electrode 223 is electrically connected to another end of the electrically conductive heat generation structure 222, and another end of the second electrode 223 extends to the outer surface of the housing 1. A positive electrode of the power storage battery module 9 is electrically connected to the first electrode 221, and a negative electrode of the power storage battery module 9 is electrically connected to the second electrode 223. Ends of the first electrode 221 and the second electrode 223 extending to the outer surface of the housing 1 are configured to connect to the power supply module. After the ends are connected to the power supply module, a loop is formed by the power supply module, the first electrode 221, the electrically conductive heat generation structure 222 and the second electrode 223. When current passes through the electrically conductive heat generation structure 222, heat is generated and temperature rises in the electrically conductive heat generation structure 222, thereby heating the atomization liquid. Meanwhile, when current flows through the power storage battery module 9 via the first electrode 221 and the second electrode 223, the power storage battery module 9 stores electrical energy.

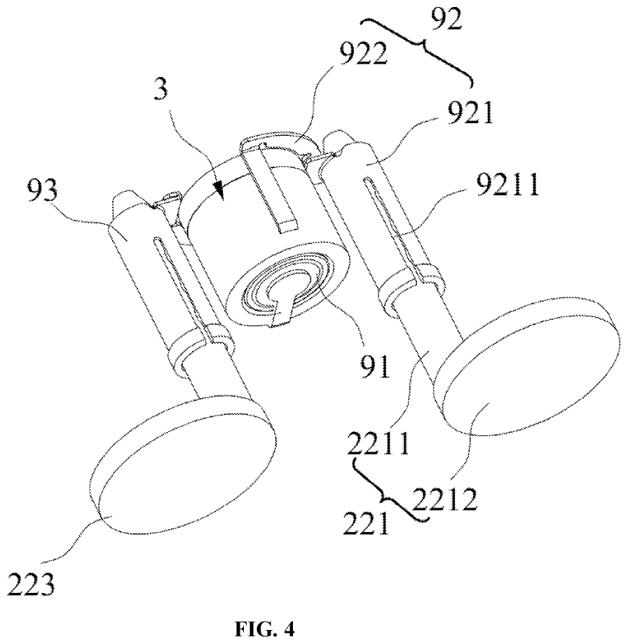

Specifically, as shown in FIG. 4, the power storage battery module 9 includes a power storage battery 91, a first connection terminal 92 and a second connection terminal 93. The power storage battery 91 is electrically connected to the functional module 3. One end of the first connection terminal 92 is electrically connected to the first electrode 221, and another end of the first connection terminal 92 is electrically connected to a positive electrode of the power storage battery 91. One end of the second connection terminal 93 is electrically connected to the second electrode 223, and another end of the second connection terminal 93 is electrically connected to a negative electrode of the power storage battery 91. The first connection terminal 92 achieves electrical connection between the first electrode 221 and the positive electrode of the power storage battery 91, and the second connection terminal 93 achieves electrical connection between the second electrode 223 and the negative electrode of the power storage battery 91. The power storage battery 91 is configured to store electrical energy.

More specifically, the first connection terminal 92 includes a first sleeve portion 921 and a first elastic piece 922. The first sleeve portion 921 is sleeved on the first electrode 221, one end of the first elastic piece 922 is connected to the first sleeve portion 921, and another end of the first elastic piece 922 is welded to the positive electrode of the power storage battery 91. The second connection terminal 93 includes a second sleeve portion and a second elastic piece. The second sleeve portion is sleeved on the second electrode 223, one end of the second elastic piece is connected to the second sleeve portion, and another end of the second elastic piece is welded to the negative electrode of the power storage battery 91. The first sleeve portion 921 is connected to the first electrode 221 by sleeving, which improves firmness of the electrical connection between the first sleeve portion 921 and the first electrode 221, and the first elastic piece 922 is welded to the power storage battery 91, which improves the firmness of the electrical connection between the first elastic piece 922 and the power storage battery 91. Similarly, the second sleeve portion is connected to the second electrode 223 by sleeving, which improves firmness of the electrical connection between the second sleeve portion and the second electrode 223, and the second elastic piece is welded to the power storage battery 91, which improves the firmness of the electrical connection between the second elastic piece and the power storage battery 91. The first elastic piece 922 and the second elastic piece have an elastic buffering effect, so that the electrical connections are prevented from being disconnected due to shaking, thereby further ensuring the firmness of the electrical connections.

In this embodiment, the first elastic piece 922 is welded to the power storage battery 91 by laser or infrared means; and the second elastic piece is welded to the power storage battery 91 by laser or infrared means.

Further specifically, an outer sidewall of the first sleeve portion 921 is provided with multiple first through slots 9211 in communication with the inside of the first sleeve portion 921, one end of each of the first through slots 9211 extends to one end of the first sleeve portion 921, and the first elastic piece 922 is connected to another end of the first sleeve portion 921. An outer sidewall of the second sleeve portion is provided with multiple second through slots in communication with the inside of the second sleeve portion, one end of each of the second through slots extends to one end of the second sleeve portion, and the second elastic piece is connected to another end of the second sleeve portion. The first through slots 9211 are provided so that the sidewall of the first sleeve portion 921 can be elastically deformed in a certain degree, and in this way, the first sleeve portion 921 can clamp the first electrode 221 tightly. Similarly, the second through slots are provided so that the sidewall of the second sleeve portion can be elastically deformed in a certain degree, and in this way, the second sleeve portion can clamp the second electrode 223 tightly.

Specifically, the functional module 3 is disposed between the first electrode 221 and the second electrode 223, and the power storage battery 91 is disposed in an installation recess at one end of the functional module 3. With this arrangement, the electrical connection line between the functional module 3 and the power storage battery 91 is made short, and the distance between the first electrode 221 and the power storage battery 91 and the distance between the second electrode 223 and the power storage battery 91 are both made small, so that the lengths of the first connection terminal 92 and the second connection terminal 93 are both made small, which is advantageous for reducing the cost of the material. Furthermore, the structure is compact, the occupied space is small, and the increase of the volume of the atomization device due to the increase of the power storage battery module 9 is avoided.

Specifically, as shown in FIGS. 1, 3, and 4, the first electrode 221 includes a first connecting post 2211 and a first contact piece 2212 connected to one end of the first connecting post 2211, one end, facing away from the first contact piece 2212, of the first connecting post 2211 is electrically connected to the electrically conductive heat generation structure 222, the first contact piece 2212 is embedded on the outer surface of the housing 1, and the first connecting post 2211 is electrically connected to the positive electrode of the power storage battery module 9. The second electrode 223 includes a second connecting post and a second contact piece connected to one end of the second connecting post, one end, facing away from the second contact piece, of the second connecting post is electrically connected to the electrically conductive heat generation structure 222, the second contact piece is embedded on the outer surface of the housing 1, and the second connecting post is electrically connected to the negative electrode of the power storage battery module 9. The first contact piece 2212 and the second contact piece increase the area of contact with the power supply module to facilitate the power supply of the power supply module to the atomization device.

With continued reference to FIGS. 1, 3, and 4, in this embodiment, the first sleeve portion 921 is sleeved on the first connecting post 2211, one end, on which the first through slot 9211 is provided, of the first sleeve portion 921 is disposed adjacent to the first contact piece 2212, one end, facing away from the first contact piece 2212, of the first connecting post 2211 is provided with a conical head, and the conical head facilitates inserting the first connecting post 2211 into the first sleeve portion 921 and facilitates the fitting of the first electrode 221 to the first connection terminal 92. Similarly, the second sleeve portion is sleeved on the second connecting post, one end, on which the second through slot is provided, of the second sleeve portion is disposed adjacent to the second contact piece, one end, facing away from the second contact piece, of the second connecting post is provided with a conical head, and the conical head facilitates inserting the second connecting post into the second sleeve portion and facilitates the fitting of the second electrode 223 to the second connection terminal 93.

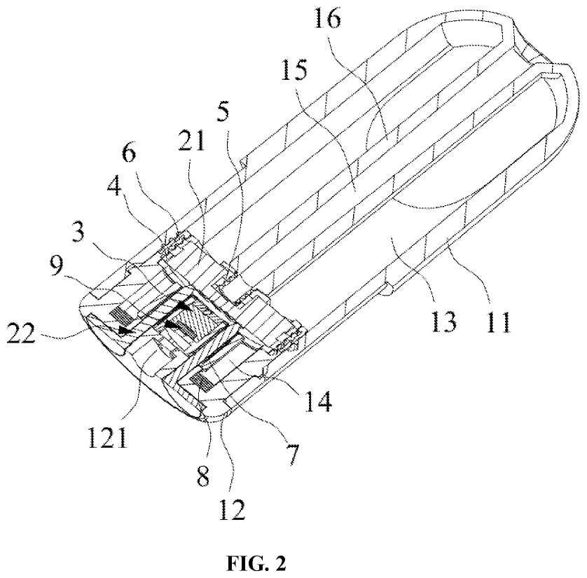

Specifically, as shown in FIG. 2, the atomization module 2 further includes a liquid infiltration member 21. The atomization liquid is disposed on one side of the liquid infiltration member 21, and the electrically conductive heating circuit 22 is connected on another side of the liquid infiltration member 21. The liquid infiltration member 21 is capable of being infiltrated by the atomization liquid. The atomization liquid infiltrates into the liquid infiltration member 21, and the electrically conductive heating circuit 22 heats up when energized to heat and atomize the atomization liquid in the liquid infiltration member 21 to implement atomization.

In this embodiment, the liquid infiltration member 21 is made of a food-grade environment-friendly high-temperature-resistant porous composite material.

Specifically, as shown in FIGS. 1 and 2, the housing 1 includes a main body housing 11 and a housing cover 12, and the housing cover 12 is covered on the main body housing 11. One end of the liquid infiltration member 21 is disposed in the housing cover 12, and another end of the liquid infiltration member 21 is disposed in the main body housing 11. A storage chamber 13 for storing the atomization liquid is disposed on a side, facing the main body housing 11, of the liquid infiltration member 21, and an atomization chamber 14 is disposed on a side, facing the housing cover 12, of the liquid infiltration member 21. Each of the electrically conductive heating circuit 22, the functional module 3, and the power storage battery module 9 is disposed in the atomization chamber 14. The liquid infiltration member 21 is blocked between the storage chamber 13 and the atomization chamber 14, by which, not only the atomization liquid in the storage chamber 13 is prevented from flowing into the atomization chamber 14, but also heating and atomizing of the atomization liquid by the electrically conductive heating circuit 22 can be realized. The main body housing 11 and the housing cover 12 are connected to each other by covering on each other so as to facilitate the placement of the components and the atomization liquid in the housing 1.

In this embodiment, both the main body housing 11 and the housing cover 12 are made of a food-grade environment-friendly material.

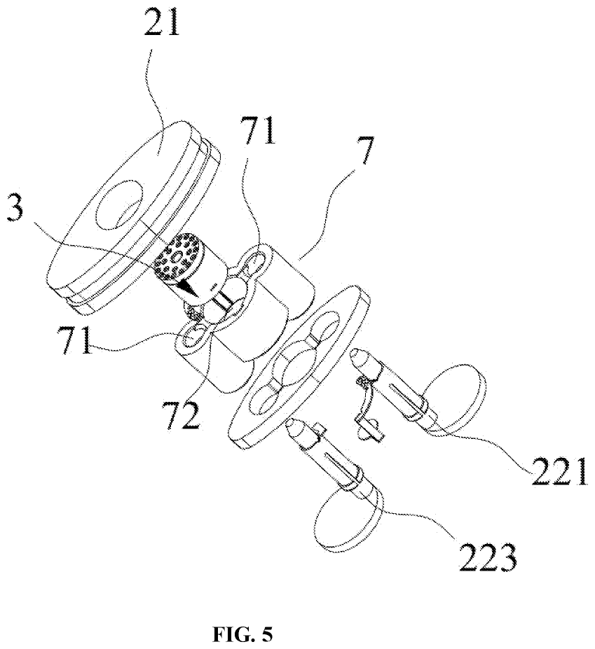

Specifically, as shown in FIGS. 2 and 5, a fixation holder 7 is disposed in the atomization cavity 14, the fixation holder 7 is provided with two limiting holes 71 and a limiting recess 72 disposed between the two limiting holes 71 at intervals, the functional module 3 is disposed in the limiting recess 72, and the first electrode 221 and the second electrode 223 are respectively inserted in the two limiting holes 71. The fixation holder 7 enables positioning of the functional module 3 and the first electrode 221 and the second electrode 223.

Specifically, a cylinder 16 extending in a fitting direction of the main body housing 11 and the housing cover 12 is disposed in the storage chamber 13, one end of the cylinder 16 is connected to an end wall of the storage chamber 13, and another end of the cylinder 16 is provided to be inserted into the liquid infiltration member 21. The cylinder 16 is provided with a gas channel 15 passing through the cylinder 16 in the length direction of the cylinder 16, and a gas outlet 121 in communication with the outside is provided in the atomization chamber 14. The gas outside can enter the atomization chamber 14 through the gas channel 15 to equalize the gas pressure in the atomization chamber 14 and the gas pressure of outside so as to ensure that the atomization vapor, formed by atomizing, in the atomization chamber 14 can get out through the gas outlet 121.

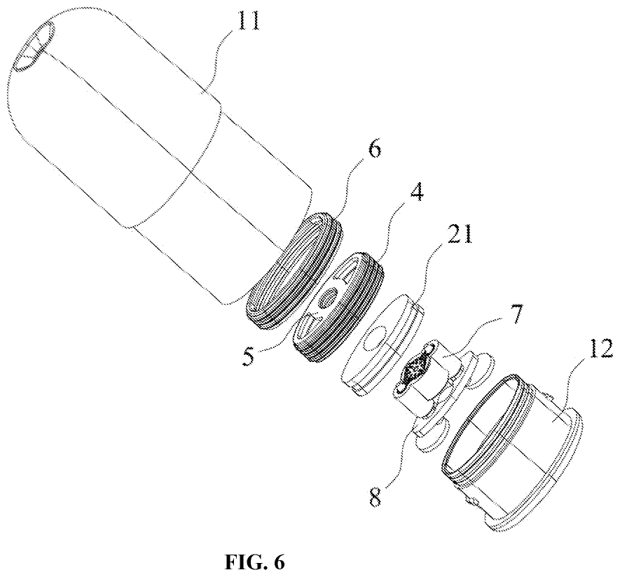

Specifically, as shown in FIGS. 2 and 6, a first sealing ring 4 is disposed between a circumferential outer wall of the liquid infiltration member 21 and a circumferential inner wall of the housing cover 12, for sealing, and the first sealing ring 4 improves the airtightness of the connection between the liquid infiltration member 21 and the housing cover 12. A second sealing ring 5 is disposed between an inner wall of the liquid infiltration member 21 and an outer wall of the cylinder 16, for sealing, and the second sealing ring 5 improves the airtightness of the connection between the liquid infiltration member 21 and the cylinder 16. A third sealing ring 6 is disposed between the circumferential outer wall of the liquid infiltration member 21 and a circumferential inner wall of the main body housing 11, for sealing, and the third sealing ring 6 improves the airtightness of the connection between the liquid infiltration member 21 and the main body housing 11. With this arrangement, the airtightness between the storage chamber 13 and the atomization chamber 14 is ensured, so that the atomization liquid in the storage chamber 13 is prevented from flowing into the atomization chamber 14, thereby ensuring normal operation of the components in the atomization chamber 14.

Specifically, as shown in FIG. 6, the first sealing ring 4 and the second sealing ring 5 are connected to each other and integrally formed, which improves the manufacturing efficiency and the sealing effect.

More specifically, an inner surface and an outer surface of each of the first sealing ring 4, the second sealing ring 5 and the third sealing ring 6 are both corrugated surfaces, which further improves the sealing performance.

In this embodiment, the first sealing ring 4, the second sealing ring 5, and the third sealing ring 6 are all made of food-grade environment-friendly silica gel.

Specifically, as shown in FIG. 2, the third sealing ring 6 is disposed between the first sealing ring 4 and the circumferential inner wall of the main body housing 11, for sealing.

Specifically, as shown in FIGS. 2 and 6, a residual liquid adsorbing layer 8 is laid on an inner end wall of one end, facing away from the storage chamber 13, of the atomization chamber 14. The atomization vapor formed by atomization through the atomization module 2 is dispersed in the atomization chamber 14. The atomization vapor in the atomization chamber 14 is discharged through the gas outlet 121 due to the effect of the gas pressure difference when the gas pressure at the gas outlet 121 is reduced. During use, a small amount of atomization vapor is liquefied in the atomization chamber 14, and the residual liquid adsorbing layer 8 is configured to adsorb the liquid formed by the liquifying to prevent the liquid from adversely affecting the normal operation of the components.

In this embodiment, the residual liquid adsorbing layer 8 is made of food-grade environment-friendly condensed fibers.

Specifically, the gas component detection unit, a blood oxygen detection unit and a heart rate detection unit are integrated in the functional module 3. The functional module 3 is provided such that the atomization device can detect the composition of the gas, the blood oxygen concentration and the heart rate. By detecting the composition of the surrounding gas, it is known whether the gas contains harmful gas, thereby reminding the user to stay away from the gas in time, which facilitates guaranteeing personal safety. By detecting the blood oxygen concentration and the heart rate, the physical condition of the user is obtained, so that the physical health condition can be known in time, and medical treatment can be performed in a timely manner, thereby avoiding further aggravation of symptoms.

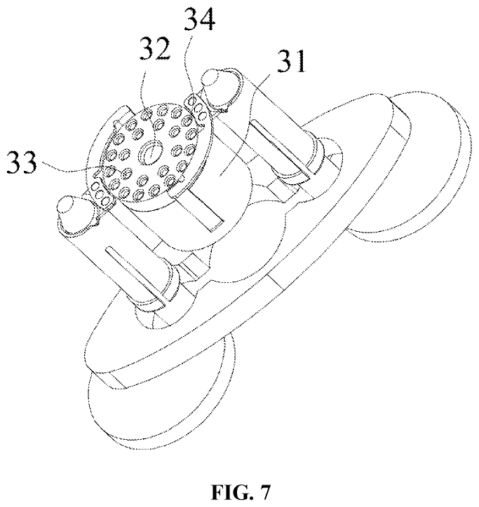

More specifically, as shown in FIGS. 1 and 7, the functional module 3 includes a block 31 disposed in the atomization chamber 14, an end surface of an end, facing the gas channel 15, of the block 31 is a detection surface, and each of the blood oxygen detection unit, the heart rate detection unit and the gas component detection unit is disposed on the detection surface. The outside gas enters the atomization chamber 14 through the gas channel 15 and flows directly through the detection surface. The gas component detection unit detects the gas to obtain components of the gas. The blood oxygen detection unit and the heart rate detection unit emit light outwardly through the gas channel 15 to obtain the blood oxygen concentration and the heart rate of the user.

Further specifically, a first mounting hole is formed in the center of the detection surface, multiple second mounting holes arranged in one circle and multiple third mounting holes arranged in one circle are disposed in the detection surface with the first mounting hole as the center, and the third mounting holes are disposed in an outer circle of the second mounting holes. The heart rate detection unit includes a heart rate detection member 32 disposed in the first mounting hole. The blood oxygen detection unit includes multiple blood oxygen detection members 33 disposed in the multiple second mounting holes in one-to-one correspondence. The gas composition detection unit includes multiple gas detection members 34 disposed in the multiple third mounting holes in one-to-one correspondence. According to this arrangement, the gas component detection unit, the blood oxygen detection unit and the heart rate detection unit are integrated in the functional module 3, so that multiple functions are all realized, the functional diversity of the atomization device is increased, to enable the atomization device to have multiple usages, and improve the user experience. Moreover, the distribution positions of the heart rate detection member 32, the blood oxygen detection member 33, and the gas detection member 34 on the detection surface all improve the detection accuracy.

In this embodiment, an end face of an end, facing away from the detection surface, of the block 31 is provided with an installation recess for accommodating the power storage battery 91. The end provided with the installation recess faces a recess bottom of the limiting recess 72 of the fixation holder 7 and is disposed in the limiting recess 72, to reliably fix the power storage battery 91.

Specifically, the gas detection member 34 includes a sensor for monitoring nicotine, PM2.5, formaldehyde, heavy metal, carbon monoxide, and the like in the air in real time. When outside gas passes by the contact surface through the gas channel 15, the gas detection member 34 detects the composition of the gas. For the specific composition and detection principle of the gas detection member 34, reference may be made to the conventional technology, which is not described in detail herein.

Specifically, the heart rate detection member 32 obtains the heart rate of the user by emitting green light to the outside and the green light being shot into the skin of the user. For the specific composition and the detection principle of the heart rate detection member 32, reference can be made to the conventional technology, which is not described in detail herein.

Specifically, the blood oxygen detection member 33 obtains the blood oxygen concentration of the user by emitting the infrared rays and red light to the outside and the infrared rays and red light being shot into the skin of the user. For the specific composition and the detection principle of the blood oxygen detection member 33, reference can be made to the conventional technology, which is not described in detail herein.

Specifically, the functional module 3 further includes a Bluetooth or Wi-Fi transmitting module, and the functional module 3 can have the data measured by it connected to the APP software on the mobile phone through the Bluetooth or Wi-Fi transmitting module, and perform data analysis and summarization, and automatically generate an environment and health examination report, so that the environment and the physical condition can be timely and more clearly understood.

An electronic cigarette is further provided according to this embodiment, which includes a power supply module (not shown) and the atomization device as described above, the power supply module is detachably connected to the housing 1 and can supply power to the electrically conductive heating circuit 22.

When to use the electronic cigarette, the power supply module is connected to the housing 1 of the atomization device, and at this time, the two terminals of the power supply module are in contact with the first contact piece 2212 and the second contact piece of the atomization device, respectively, so as to implement electrical conduction. The electrically conductive heating circuit 22 heats the atomization liquid in the liquid infiltration member 21, and the atomization vapor formed by the atomization is discharged through the gas outlet 121 and enters the mouth of the user. In the process of smoking, the power storage battery 91 is automatically charged. Therefore, when the electronic cigarette is not smoked, the power storage battery 91 can supply power to the functional module 3. Therefore, the functional module 3 of the electronic cigarette can independently operate, so that the electronic cigarette can be used flexibly and variously, and the user experience is improved.

Apparently, the preceding embodiments of the present disclosure are only examples made for clearly illustrating the present disclosure rather than limiting the implementations of the present disclosure. For the person of ordinary skill in the art, changes or alterations in other different forms may also be made based on the preceding description. It is not necessary and impossible to exhaust all implementations herein. Any modifications, equivalent substitutions, and improvements made within the spirit and principle of the present disclosure fall within the scope of the claims of the present disclosure.

What is claimed is:

1. An atomization device, comprising:
a housing, wherein an atomization liquid is stored in the housing;
an atomization module disposed within the housing, wherein the atomization module comprises an electrically conductive heating circuit, and the electrically conductive heating circuit is capable of heating and atomizing the atomization liquid while the electrically conductive heating circuit is energized;
a functional module comprising a gas component detection unit;
a power storage battery module electrically connected to the functional module and capable of supplying power to the functional module, wherein the power storage battery module is electrically connected to the electrically conductive heating circuit, and while the electrically conductive heating circuit is energized, the power storage battery module is charged.

2. The atomization device according to claim 1, wherein the electrically conductive heating circuit comprises a first electrode, an electrically conductive heat generation structure and a second electrode, one end of the first electrode is electrically connected to one end of the electrically conductive heat generation structure, another end of the first electrode extends to an outer surface of the housing, one end of the second electrode is electrically connected to another end of the electrically conductive heat generation structure, another end of the second electrode extends to the outer surface of the housing, a positive electrode of the power storage battery module is electrically connected to the first electrode, and a negative electrode of the power storage battery module is electrically connected to the second electrode.

3. The atomization device according to claim 2, wherein the power storage battery module comprises a power storage battery, a first connection terminal and a second connection terminal, the power storage battery is electrically connected to the functional module, one end of the first connection terminal is electrically connected to the first electrode, and another end of the first connection terminal is electrically connected to a positive electrode of the power storage battery, one end of the second connection terminal is electrically connected to the second electrode, and another end of the second connection terminal is electrically connected to a negative electrode of the power storage battery.

4. The atomization device according to claim 3, wherein the first connection terminal comprises a first sleeve portion and a first elastic piece, the first sleeve portion is sleeved on the first electrode, one end of the first elastic piece is connected to the first sleeve portion, and another end of the first elastic piece is welded to the positive electrode of the power storage battery; and
the second connection terminal comprises a second sleeve portion and a second elastic piece, the second sleeve portion is sleeved on the second electrode, one end of the second elastic piece is connected to the second sleeve portion, and another end of the second elastic piece is welded to the negative electrode of the power storage battery.

5. The atomization device according to claim 4, wherein an outer sidewall of the first sleeve portion is provided with a plurality of first through slots in communication with the inside of the first sleeve portion, one end of each of the first through slots extends to an end portion of one end of the first sleeve portion, and the first elastic piece is connected to another end of the first sleeve portion; and
an outer sidewall of the second sleeve portion is provided with a plurality of second through slots in communication with the inside of the second sleeve portion, one end of each of the second through slots extends to an end portion of one end of the second sleeve portion, and the second elastic piece is connected to another end of the second sleeve portion.

6. The atomization device according to claim 2, wherein the first electrode comprises a first connecting post and a first contact piece connected to one end of the first connecting post, one end, facing away from the first contact piece, of the first connecting post is electrically connected to the electrically conductive heat generation structure, the first contact piece is embedded on the outer surface of the housing, and the first connecting post is electrically connected to the positive electrode of the power storage battery module; and
the second electrode comprises a second connecting post and a second contact piece connected to one end of the second connecting post, one end, facing away from the second contact piece, of the second connecting post is electrically connected to the electrically conductive heat generation structure, the second contact piece is embedded on the outer surface of the housing, and the second connecting post is electrically connected to the negative electrode of the power storage battery module.

7. The atomization device according to claim 1, wherein the atomization module further comprises a liquid infiltration member, the atomization liquid is disposed on one side of the liquid infiltration member, the electrically conductive heating circuit is connected to another side of the liquid infiltration member, and the liquid infiltration member is capable of being infiltrated by the atomization liquid.

8. The atomization device according to claim 7, wherein the housing comprises a main body housing and a housing cover, and the housing cover is covered on the main body housing, one end of the liquid infiltration member is disposed in the housing cover, and another end of the liquid infiltration member is disposed in the main body housing, a storage chamber for storing the atomization liquid is disposed on a side, facing the main body housing, of the liquid infiltration member, and an atomization chamber is disposed on a side, facing the housing cover, of the liquid infiltration member, each of the electrically conductive heating circuit, the functional module, and the power storage battery module is disposed in the atomization chamber.

9. The atomization device according to claim 1, wherein the gas component detection unit, a blood oxygen detection unit and a heart rate detection unit are integrated in the functional module.

10. An electronic cigarette, comprising a power supply module, further comprising the atomization device according to claim 1, wherein the power supply module is detachably connected to the housing and is capable of supplying power to the electrically conductive heating circuit.

11. The atomization device according to claim 2, wherein the gas component detection unit, a blood oxygen detection unit and a heart rate detection unit are integrated in the functional module.

12. The atomization device according to claim 3, wherein the gas component detection unit, a blood oxygen detection unit and a heart rate detection unit are integrated in the functional module.

13. The atomization device according to claim 4, wherein the gas component detection unit, a blood oxygen detection unit and a heart rate detection unit are integrated in the functional module.

14. The atomization device according to claim 5, wherein the gas component detection unit, a blood oxygen detection unit and a heart rate detection unit are integrated in the functional module.

15. The atomization device according to claim 6, wherein the gas component detection unit, a blood oxygen detection unit and a heart rate detection unit are integrated in the functional module.

16. The atomization device according to claim 7, wherein the gas component detection unit, a blood oxygen detection unit and a heart rate detection unit are integrated in the functional module.

17. The atomization device according to claim 8, wherein the gas component detection unit, a blood oxygen detection unit and a heart rate detection unit are integrated in the functional module.

18. The electronic cigarette according to claim 10, wherein the electrically conductive heating circuit comprises a first electrode, an electrically conductive heat generation structure and a second electrode, one end of the first electrode is electrically connected to one end of the electrically conductive heat generation structure, another end of the first electrode extends to an outer surface of the housing, one end of the second electrode is electrically connected to another end of the electrically conductive heat generation structure, another end of the second electrode extends to the outer surface of the housing, a positive electrode of the power storage battery module is electrically connected to the first electrode, and a negative electrode of the power storage battery module is electrically connected to the second electrode.

19. The electronic cigarette according to claim 18, wherein the power storage battery module comprises a power storage battery, a first connection terminal and a second connection terminal, the power storage battery is electrically connected to the functional module, one end of the first connection terminal is electrically connected to the first electrode, and another end of the first connection terminal is electrically connected to a positive electrode of the power storage battery, one end of the second connection terminal is electrically connected to the second electrode, and another end of the second connection terminal is electrically connected to a negative electrode of the power storage battery.

20. The electronic cigarette according to claim 19, wherein the first connection terminal comprises a first sleeve portion and a first elastic piece, the first sleeve portion is sleeved on the first electrode, one end of the first elastic piece is connected to the first sleeve portion, and another end of the first elastic piece is welded to the positive electrode of the power storage battery; and
the second connection terminal comprises a second sleeve portion and a second elastic piece, the second sleeve portion is sleeved on the second electrode, one end of the second elastic piece is connected to the second sleeve portion, and another end of the second elastic piece is welded to the negative electrode of the power storage battery.
